SEQUENCE LISTING (29 sequences)

SEQ ID NO: 1 - DNA, 56 bp, Artificial Sequence
  Synthetic
accggcctttcactactcctacttcaagagagtaggagtagtgaaaggcctttttc

SEQ ID NO: 2 - DNA, 56 bp, Artificial Sequence
  Synthetic
tgcagaaaaaggcctttcactactcctactctcttgaagtaggagtagtgaaaggc

SEQ ID NO: 3 - DNA, 24 bp, Artificial Sequence
  Synthetic
atcctaaggaccagcttctttggg

SEQ ID NO: 4 - DNA, 51 bp, Artificial Sequence
  Synthetic
atcctgccgagacggttacgctcacgcgtctcagagatcttgggcctctgc

SEQ ID NO: 5 - DNA, 24 bp, Artificial Sequence
  Synthetic
atcctaaggaccagcttctttggg

SEQ ID NO: 6 - DNA, 89 bp, Artificial Sequence
  Synthetic
atctctacttttgaacgtaggagtagtgaaaggccagagaacttggcctttcactactcctacagatcttggggcctctgccccgacac

SEQ ID NO: 7 - DNA, 24 bp, Artificial Sequence
  Synthetic
atcctaaggaccagcttctttggg

SEQ ID NO: 8 - DNA, 96 bp, Artificial Sequence
  Synthetic
atccagtctcattttgaaactccagaaagtggcctttcactactcctactgacaggaaggtaggagtagtgaaaggccgagatcttgggcctctgc

SEQ ID NO: 9 - DNA, 24 bp, Artificial Sequence
  Synthetic
atcctaaggaccagcttctttggg

SEQ ID NO: 10 - DNA, 89 bp, Artificial Sequence
  Synthetic
atccagtctcattttgaaacctcggcctttcactactcctactgacaggaaggtaggagtagtgaaaggccgagatcttgggcctctgc

SEQ ID NO: 11 - DNA, 24 bp, Artificial Sequence
  Synthetic
atcctaaggaccagcttctttggg

SEQ ID NO: 12 - DNA, 98 bp, Artificial Sequence
  Synthetic
atccagtctcattttgaaactccagaaagtaaggcctttcactactcctactgacaggaaggtaggagtagtgaaaggccgagatcttgggcctctgc

SEQ ID NO: 13 - DNA, 51 bp, Artificial Sequence
  Synthetic
accggcctttcactactcctacttcaagagagtaggagtagtgaaaggccc

SEQ ID NO: 14 - DNA, 51 bp, Artificial Sequence
  Synthetic
tgcagggcctttcactactcctactctcttgaagtaggagtagtgaaaggc

SEQ ID NO: 15 - DNA, 24 bp, Artificial Sequence
  Synthetic
atcctaaggaccagcttctttggg

SEQ ID NO: 16 - DNA, 79 bp, Artificial Sequence
  Synthetic
tactccactgcagggcctttcactactcctactgacaggaaggtaggagtagtgaaaggccgagatcttgggcctctgc

SEQ ID NO: 17 - DNA, 120 bp, Artificial Sequence
  Synthetic
accggcctttcactactcctacttcaagagagtaggagtagtgaaaggcctttttctgcatggccggaaagtgatgaggatgaagttctctcatcctcatcactttccggaaaaagacgt

SEQ ID NO: 18 - DNA, 104 bp, Artificial Sequence
  Synthetic
cggggcagaggcccaagatctctgagacgcgtgagcgtaaccgtctcggcaggccccgtctccgggttctagagactctgcgcactcgcattggcagagccgtc

SEQ ID NO: 19 - DNA, 82 bp, Artificial Sequence
  Synthetic
cggggcagaggccccaagatctgtaggagtagtgaaaggccaagttctctggcctttcactactcctacgttcaaaagtaga

SEQ ID NO: 20 - DNA, 97 bp, Artificial Sequence
  Synthetic
cggggcagaggcccaagatctcggcctttcactactcctaccttcctgtcagtaggagtagtgaaaggccactttctggagtttcaaaagtagactg

SEQ ID NO: 21 - DNA, 90 bp, Artificial Sequence
  Synthetic
cggggcagaggcccaagatctcggcctttcactactcctaccttcctgtcagtaggagtagtgaaaggccgaggtttcaaaagtagactg

SEQ ID NO: 22 - DNA, 99 bp, Artificial Sequence
  Synthetic
cggggcagaggcccaagatctcggcctttcactactcctaccttcctgtcagtaggagtagtgaaaggccttactttctggagtttcaaaagtagactg

SEQ ID NO: 23 - DNA, 51 bp, Artificial Sequence; identical to SEQ ID NO: 13
  Synthetic

SEQ ID NO: 24 - DNA, 80 bp, Artificial Sequence
  Synthetic
cggggcagaggcccaagatctcggcctttcactactcctaccttcctgtcagtaggagtagtgaaaggccctgcagtgga

SEQ ID NO: 25 - DNA, 429 bp, Artificial Sequence
  Synthetic
atcctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctctgagacgcgtgagcgtaaccgtctcggcagatg

SEQ ID NO: 26 - DNA, 101 bp, Artificial Sequence
  Synthetic
ctcgggagtgcgcggggcactctatcattgatagagtaagtgaccgtgtgtgtaaagagtgaggcgtatgaggctgtgtcggggcagaggcccaagatctc

SEQ ID NO: 27 - DNA, 107 bp, Artificial Sequence
  Synthetic
ctcgggagtgcgcggggcctccctatcagtgatagagaaagtgaagtgaccgtgtgtgtaaagagtgaggcgtatgaggctgtgtcggggcagaggcccaagatctc

SEQ ID NO: 28 - DNA, 76 bp, Artificial Sequence
  Synthetic
ctcgggagtgcgcggggcaagtgaccgtgtgtgtaaagagtgaggcgtatgaactctatcattgatagagtatctc

SEQ ID NO: 29 - DNA, 82 bp, Artificial Sequence
  Synthetic
ctcgggagtgcgcggggcaagtgaccgtgtgtgtaaagagtgaggcgtatgactccctatcagtgatagagaaagtgatctc

(19) United States
(12) Patent Application Publication
(10) Pub. No.: US 2006/0040391 A1
(43) Pub. Date: Feb. 23, 2006

(54) RNA interference vectors

(75) Inventors: Cheryl Bailey, Cedar Bluffs, NE (US); Doug Storts, Verona, WI (US); Martin Rosenberg, Madison, WI (US)

Correspondence Address: David A. Casimir;MEDLEN & CARROLL, LLP, Suite 350, 101 Howard Street, San Francisco, CA, 94105, US

(73) Assignee: Promega Corporation, Madison, WI (US)

(21) Appl. No.: 10/922,530

(22) Filed: Aug. 20, 2004

(51) Int. Cl. A61K 48/00; C12N 15/87; C07H 21/02

(52) U.S. Cl. 435/455; 514/44; 536/23.1

(57) ABSTRACT

The present invention relates to gene-specific silencing through RNA interference, and in particular, to vectors for expressing RNAi molecules. In some embodiments, the present invention provides compositions and methods for inducible expression of RNAi molecules, and/or for long-term expression of RNAi molecules. Hence the compositions and methods described herein are suitable for regulatable and/or sustained gene-specific silencing in cells.

### FIELD OF INVENTION

The present invention relates to gene-specific silencing through RNA interference, and in particular, to vectors for expressing RNAi molecules. In some embodiments, the present invention provides compositions and methods for inducible expression of RNAi molecules, and/or for long-term expression of RNAi molecules. Hence the compositions and methods described herein are suitable for regulatable and/or sustained gene-specific silencing in cells.

### BACKGROUND OF THE INVENTION

Double-stranded RNA interference (RNAi) is a powerful means for selectively silencing gene expression in eukaryotes (Sharp, Genes Dev., 15:485-490, 2001; and Elbashir et al., Genes Dev., 15:188-200, 2001). In mammalian cells, gene-specific silencing can be accomplished in one of two ways. In the first, short interfering RNAs (siRNAs) are synthesized in vitro and directly transfected into cells to achieve transient suppression of gene expression. In the second, short hairpin RNAs (shRNAs) are transcribed in vivo from an RNAi vector (Yu et al., Proc Natl Acad Sci USA, 99:6047-6052, 2002; Sui et al., Proc Natl Acad Sci USA, 99:5515-5520, 2002; and Brummelkamp et al., Science, 296:550-553, 2002). The latter method is desirable in that gene-specific RNAi vectors are relatively inexpensive to construct, and can be stably introduced into cells in the form of selectable plasmids or retroviruses.

Most RNAi vectors constitutively express shRNAs under the control of promoters containing an RNA polymerase III (polIII) transcription unit (e.g., H1 and U6). RNAi polIII vectors are particularly useful for shRNA expression since they are active in all tissues, and because they utilize a short T rich transcription termination site that leads to the addition of 2 bp UU overhangs (as opposed to a polyA tail) to the shRNAs. Although these vectors have been used successfully to create siRNA transgenic or “gene knockdown” mice (Carmell et al., Nature Struct Biol, 10:91-92, 2003; and Kunath et al., Nature Biotechnol, 21:559-561, 2003), they are difficult to use for achieving inducible or tissue-specific RNAi. This is particularly problematic when the target under investigation is an essential gene (required for cell survival).

Thus what is needed in the art are RNAi vectors that can be employed to suppress gene expression in an inducible and/or tissue-specific manner as well as vectors that provide other desired, enhanced expression properties.

### SUMMARY OF THE INVENTION

The present invention relates to gene-specific silencing through RNA interference, and in particular, to vectors for expressing RNAi molecules. In some embodiments, the present invention provides compositions and methods for inducible expression of RNAi molecules, and/or for long-term expression of RNAi molecules. Hence the compositions and methods described herein are suitable for regulatable and/or sustained gene-specific silencing in cells.

For example, in some embodiments, the present invention provides a composition (e.g., kit, cell, reaction mixture, etc.) comprising a vector, the vector comprising an snRNA pol II promotor operably associated with an RNAi molecule. In preferred embodiments, the promoter is a U1 or U2 promoter. In other embodiments, the promoter is a U4 or U5 promoter. The vector may further contain other promoter sequences or any other sequences common to vectors and expression vectors (e.g., restriction cloning sites, terminators, selectable marker genes (e.g., puromycin, hygromycin, and neomycin), etc.). The present invention is not limited by the nature of the RNAi molecule contained in the vector. In some embodiments, the RNAi molecule is an siRNA or an miRNA (e.g., in precursor form); see e.g., Lee, Nature, 425:415 (2003) for discussion of RNAi. In some preferred embodiments, the vector further comprises an snRNA pol II termination sequence. In some such embodiments, a spacer sequence (e.g., having 5 or more bases, e.g., 7, 10, . . . ) is located between the promoter and the termination sequence (e.g., between the RNAi molecule and the termination sequence). In some embodiments, a multicloning site is located between the promoter and the termination sequence.

An additional embodiment of the present invention provides an expression vector that is viral in origin that comprises an snRNA pol II promoter operably associated with a RNAi molecule. Examples of viral vector expression systems for mammalian systems include, but are not limited to, lentivirus, Sindbis virus, adenovirus, adeno-associated virus, and retrovirus. Examples of viral expression systems for plant systems include, but are not limited to, geminiviruses, tomato gold mosaic virus, and cauliflower mosaic virus.

The present invention also provides host cells comprising the vectors of the present invention. The present invention is not limited by the nature of the host. Host cells include, but are not limited to prokaryotic and eukaryotic cells, cells residing in culture, cells residing in tissues, and cells residing in vivo in living organisms (e.g., plants, animals, etc.). In some embodiments, the vector is stably integrated into the genome of a host cell. In other embodiments, the vector is transiently transfected into the host cell.

The present invention further comprises methods of using the vectors of the present invention. The vectors find use in the broad array of gene silencing methods for research, diagnostics, drug discovery, and therapeutics (see e.g., Prawitt et al., Cytogenet Genome Res., 105:412, 2004; Berkhout, Curr. Opin. Mol. Ther., 6:141, 2004; Downward, BMJ, 328:1245, 2004; Horiguchi, Differentiation, 72:65, 2004, each of which is herein incorporated by reference in its entirety).

The present invention further provides kits. In some embodiments, the kits provide components that permit the generation of vectors containing RNAi molecules via an amplification or extension process. For example, the present invention provides kits for cloning an RNAi molecule, comprising: i) an snRNA RNA polymerase II promoter template oligonucleotide and ii) a primer complementary to said template. In preferred embodiments, the kit further comprises one or more of: iii) a vector, iv) amplification reagents, and v) ligation reagents. In some preferred embodiments the vector is linearized and blunt ended and/or treated with a phosphatase. In some embodiments, the kit further comprises an RNAi molecule (e.g., as a positive control). In preferred embodiments, the amplification reagents comprise a high fidelity proof reading DNA polymerase (e.g., Tli polymerase), although the present invention is not limited by the nature of the polymerase. In some embodiments, the kit comprises an amplification buffer comprising magnesium sulphite.

The present invention also provides kits comprising a vector, said vector comprising an snRNA pol II promotor operably associated with an RNAi molecule. In some embodiments, the kit comprises one or more of: ligation reagents (e.g., ligase, ligase buffer), annealing buffer, positive and/or negative control samples, and instructions for use. In preferred embodiments, the kit is configured to permits cloning via sticky ended ligation of an annealed hairpin oligonucleotide and the vector. In some embodiments, the cloning process generates a new restriction site (e.g., to allow easy identification of correctly cloned constructs).

### DESCRIPTION OF THE FIGURES

FIG. 1 graphically depicts suppression of Renilla luciferase (luc) expression achieved with an RNAi vector with a U6 promoter, and an RNAi vector with a U1 promoter, initiating expression of a Renilla luc-specific hairpin siRNA. Briefly, Renilla luc-expressing HeLa cells were transfected with siRNA hairpin constructs transcribed from either a U6 (defined by primer pair A and B) or a U1 promoter (defined by primer pair E and F). Percent inhibition is presented after normalization to cell viability and non-specific inhibition (n=8).

FIG. 2 provides a graphical comparison of suppression of Renilla luciferase (luc) expression obtained by using different U1 promoter constructs. Two vectors were transfected into Renilla luciferase expressing HeLa cells, either with (Puro) or without (Basic) a puromycin selectable gene, and each containing one of: i) a U1 promoter region only (U1 Cloning); ii) a U1 promoter and a three bp spacer between the hairpin structure and the termination box; and iii) a U1 promoter and a ten base pair spacer between the hairpin structure and the termination box. Percent inhibition is presented after normalization to cell viability and non-specific inhibition (n=8).

FIG. 3 provides a comparison of suppression levels of Renilla luciferase (luc) expression achieved over time after transient transfection of U6 and U1 promoter RNAi constructs initiating expression of a Renilla luc hairpin siRNA. HeLa cells stably expressing Renilla luc were transfected with hairpin siRNA DNA constructs containing either the U6 promoter or the U1 promoter and a 12 bp spacer between the hairpin sequence and the termination box. Data represent normalization to cell viability and non-specific hairpin inhibition (n=6).

FIG. 4 depicts the effect of termination box sequence on suppression of Renilla luciferase (luc) expression. HeLa cells stably expressing Renilla luc were transfected with hairpin Renilla luc siRNA with and without termination sequences. Cells were assayed for Renilla luc expression, which was normalized to cell viability and non-specific inhibition (n=6).

FIG. 5 panel A provides the sequence of the Renilla luciferase hairpin insert of the U6 positive control (SEQ ID NO:17) generated with oligonucleotides A and B, and panel B provides the sequence of the 3′ end of the U1 promoter control amplification product (SEQ ID NO:18) generated with primers C and D.

FIG. 6 panel A provides the sequence of the Renilla luciferase hairpin insert lacking spacer nucleotides (SEQ ID NO:19) generated with oligonucleotides E and F, panel B provides the sequence of the Renilla luciferase hairpin insert containing a 10 bp spacer (SEQ ID NO:20) generated with oligonucleotides G and H, and panel C provides the sequence of the Renilla luciferase hairpin insert containing a 3 bp spacer (SEQ ID NO:21) generated with oligonucleotides I and J.

FIG. 7 panel A provides the sequence of the Renilla luciferase hairpin insert containing a 12 bp spacer (SEQ ID NO:22) generated with oligonucleotides K and L, panel B provides the sequence of the U6 construct Renilla luciferase hairpin insert lacking termination sequences (SEQ ID NO:23) generated with oligonucleotides L and M, and panel C provides the sequence of the U1 construct Renilla luciferase hairpin insert lacking termination sequences (SEQ ID NO:24) generated with oligonucleotides N and O.

FIG. 8 shows an exemplary viral vector of the present invention.

FIG. 9 shows exemplary kit components of the present invention.

### DEFINITIONS

To facilitate an understanding of the present invention, a number of terms and phrases as used herein are defined below:

The term “gene” refers to a nucleic acid (e.g., DNA or RNA) sequence that comprises coding sequences necessary for the production of an RNA, and/or a polypeptide, or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” may also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The term “heterologous gene” refers to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise cDNA forms of the gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The terms “region” or “portion” when used in reference to a nucleic acid molecule refer to a set of linked nucleotides that is less than the entire length of the molecule.

The term “strand” when used in reference to a nucleic acid molecule refers to a set of linked nucleotides which comprises either the entire length or less than or the entire length of the molecule.

The term “links” when used in reference to a nucleic acid molecule refers to a nucleotide region which joins two other regions or portions of the nucleic acid molecule; such connecting means are typically though not necessarily a region of a nucleotide. In a hairpin RNAi molecule, such a linking region may join two other regions of the RNA molecule which are complementary to each other and which therefore can form a double stranded or duplex stretch of the molecule in the regions of complementarity; such links are usually though not necessarily a single stranded nucleotide region contiguous with both strands of the duplex stretch, and are referred to as “loops”.

The term “linker” when used in reference to a multiplex RNAi molecule refers to a connecting means that joins two or more RNAi molecules. Such connecting means are typically though not necessarily a region of a nucleotide contiguous with a strand of each RNAi molecule; the region of contiguous nucleotide is referred to as a “joining sequence.”

The term “a polynucleotide having a nucleotide sequence encoding a gene” or “a polynucleotide having a nucleotide sequence encoding a gene” or “a nucleic acid sequence encoding” a specified RNA molecule or polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

The terms “complementary” and “complementarity” refer to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. This is also of importance in efficacy of RNAi inhibition of gene expression or of RNA function.

The term “homology” when used in relation to nucleic acids refers to a degree of complementarity. There may be partial homology or complete homology (i.e., identity). “Sequence identity” refers to a measure of relatedness between two or more nucleic acids or proteins, and is given as a percentage with reference to the total comparison length. The identity calculation takes into account those nucleotide or amino acid residues that are identical and in the same relative positions in their respective larger sequences. Calculations of identity may be performed by algorithms contained within computer programs such as “GAP” (Genetics Computer Group, Madison, Wis.) and “ALIGN” (DNAStar, Madison, Wis.). A partially complementary sequence is one that at least partially inhibits (or competes with) a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence that is completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is, for example, 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.A.) 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that can hybridize (i.e., it is the complement of) the single-stranded nucleic acid sequence under conditions of low to high stringency as described above.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5×SSPE, 0.1% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

It is well known that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, calorimetric, and luminescent systems. It is not intended that the present invention be limited to any particular detection system or label.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “RNA function” refers to the role of an RNA molecule in a cell. For example, the function of mRNA is translation into a protein. Other RNAs are not translated into a protein, and have other functions; such RNAs include but are not limited to transfer RNA (tRNA), ribosomal RNA (rRNA), and small nuclear RNAs (snRNAs). An RNA molecule may have more than one role in a cell.

The term “inhibition” when used in reference to gene expression or RNA function refers to a decrease in the level of gene expression or RNA function as the result of some interference with or interaction with gene expression or RNA function as compared to the level of expression or function in the absence of the interference or interaction. The inhibition may be complete, in which there is no detectable expression or function, or it may be partial. Partial inhibition can range from near complete inhibition to near absence of inhibition; typically, inhibition is at least about 50% inhibition, or at least about 80% inhibition, or at least about 90% inhibition.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of RNA from the gene. The promoter, therefore, serves as a transcriptional regulatory element and also provides a site for initiation of transcription of the gene into RNA.

Promoters may be tissue specific or cell specific. The term “tissue specific” as it applies to a promoter refers to a promoter that is capable of directing selective expression of a nucleotide sequence of interest to a specific type of tissue in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8). A commonly used splice donor and acceptor site is the splice junction from the 16S RNA of SV40.

The term “vector” refers to nucleic acid molecules that transfer DNA segment(s) from one cell to another, and includes those nucleic acid molecules that are viral in origin. The term “vehicle” is sometimes used interchangeably with “vector.” A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from non-transfected cells, or reporter proteins, by means of which an effect on expression or activity or function of the reporter protein can be monitored.

The term “expression cassette” refers to a chemically synthesized or recombinant DNA molecule containing a desired coding sequence and appropriate nucleic acid sequences necessary for the expression of the operably linked coding sequence either in vitro or in vivo. Expression in vitro includes expression in transcription systems and in transcription/translation systems. Expression in vivo includes expression in a particular host cell and/or organism. Nucleic acid sequences necessary for expression in prokaryotic cell or in vitro expression system usually include a promoter, an operator (optional), and a ribosome binding site, often along with other sequences. Eukaryotic in vitro transcription systems and cells are known to utilize promoters, enhancers, and termination and polyadenylation signals. Nucleic acid sequences useful for expression via bacterial RNA polymerases, referred to as a transcription template in the art, include a template DNA strand which has a polymerase promoter region followed by the complement of the RNA sequence desired. In order to create a transcription template, a complementary strand is annealed to the promoter portion of the template strand. However, the present invention is not limited to any particular configuration and all known systems are contemplated.

The term “expression vector” refers to a vector comprising one or more expression cassettes. Such expression cassettes include those of the present invention, where expression results in an RNAi transcript.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, bacterial infection, viral infection, biolistics (i.e., particle bombardment) and the like. The terms “transfect” and “transform” (and grammatical equivalents, such as “transfected” and “transformed”) are used interchangeably herein.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “transgene” as used herein refers to a foreign gene that is placed into an organism by introducing the foreign gene into a cell. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

The term “host cell” refers to any cell capable of replicating and/or transcribing and/or translating a heterologous gene. Thus, a “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat. Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, β-galactosidase, alkaline phosphatase, and horse radish peroxidase.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript, i.e., at the 5′ non-coding sequence, 3′ non-coding sequence, introns, or the coding sequence. In addition, as used herein, antisense RNA may contain regions of ribozyme sequences that increase the efficacy of antisense RNA to block gene expression. “Ribozyme” refers to a catalytic RNA and includes sequence-specific endoribonucleases. “Antisense inhibition” refers to the production of antisense RNA transcripts capable of preventing the expression of the target protein.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which an RNAi molecule is homologous or complementary. Typically, when such homology or complementary is about 100%, the RNAi is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of RNAi. Such targets include unprocessed mRNA, ribosomal RNA, and viral RNA genomes.

The term “ds siRNA” refers to a siRNA molecule that comprises two separate unlinked strands of RNA that form a duplex structure, such that the siRNA molecule comprises two RNA polynucleotides.

The term “hairpin siRNA” refers to a siRNA molecule that comprises at least one duplex region where the strands of the duplex are connected or contiguous at one or both ends, such that the siRNA molecule comprises a single RNA polynucleotide. The antisense sequence, or sequence which is complementary to a target RNA, is a part of the at least one double stranded region.

The term “RNA interference” or “RNAi” refers to the silencing or decreasing of gene expression by RNAi molecules (e.g., siRNAs, miRNAs). It is the process of sequence-specific, post-transcriptional gene silencing in animals and plants, initiated by RNAi molecules that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

“MicroRNA molecules” (“miRNAs”) are small, noncoding RNA molecules that have been found in a diverse array of eukaryotes, including mammals. miRNA precursors share a characteristic secondary structure, forming short ‘hairpin’ RNAs. The term “miRNA” includes processed sequences as well as corresponding long primary transcripts (pri-miRNAs) and processed precursors (pre-miRNAs). Genetic and biochemical studies have indicated that miRNAs are processed to their mature forms by Dicer, an RNAse III family nuclease, and function through RNA-mediated interference (RNAi) and related pathways to regulate the expression of target genes (Hannon 2002, Nature 418: 244-251; Pasquinelli et al. 2002, Annu. Rev. Cell. Dev. Biol. 18: 495-513). miRNAs may be configured to permit experimental manipulation of gene expression in mammalian cells as synthetic silencing triggers ‘short hairpin RNAs’ (shRNAs) (Paddison et al. 2002, Cancer Cell 2: 17-23). Silencing by shRNAs involves the RNAi machinery and correlates with the production of small interfering RNAs (siRNAs), which are a signature of RNAi.

The term “cellular destination signal” is a portion of an RNA molecule that directs the transport of an RNA molecule out of the nucleus, or that directs the retention of an RNA molecule in the nucleus; such signals may also direct an RNA molecule to a particular subcellular location. Such a signal may be an encoded signal, or it might be added post-transciptionally.

The term “sequence-nonspecific gene silencing” refers to silencing gene expression in mammalian cells after transcription, and is induced by dsRNA of greater than about 30 base pairs. This appears to be due to an interferon response, in which dsRNA of greater than about 30 base pairs binds and activates the protein PKR and 2′,5′-oligonucleotide synthetase (2′,5′-AS). Activated PKR stalls translation by phosphorylation of the translation initiation factors eIF2alpha, and activated 2′,5′-AS causes mRNA degradation by 2′,5′-oligonucleeotide-activated ribonuclease L. These responses are intrinsically sequence-nonspecific to the inducing dsRNA.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” is used in its broadest sense. In one sense it can refer to a plant cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

### DESCRIPTION OF INVENTION

The present invention relates to gene-specific silencing through RNA interference, and in particular, to vectors for expressing RNAi molecules. In some embodiments, the present invention provides compositions and methods for inducible expression of RNAi molecules, and/or for long-term expression of RNAi molecules. Hence the compositions and methods described herein are suitable for regulatable and/or sustained gene-specific silencing in cells.

RNA interference (RNAi) is a post-transcriptional gene silencing process that is induced by a dsRNA (a small interfering RNA; siRNA), and has been used to modulate gene expression. Generally, RNAi has been performed by contacting cells with a double stranded siRNA. However, manipulation of RNA outside of cell is tedious due to the sensitivity of RNA to degradation. The present invention obviates the need for manipulating RNA by providing deoxyribonucleic acid (DNA) compositions encoding small interfering RNA (siRNA) molecules, or intermediate siRNA molecules.

miRNAs (microRNAs) are small cellular RNAs that bind to the 3′UTR, and in mammalian cells are thought to inhibit translation of a targeted message (some may mediate cleavage). They generally contain at least one mismatch to their target sequence. This is in contrast to siRNAs, which are thought to promote cleavage of mRNAs and generally do not contain mismatches to their target sequence. It appears that miRNAs may very well regulate expression of a wide variety of genes—not just genes involved in developmental and neuronal cells, although an understanding of the mechanism is not necessary to practice the present invention and the present invention is not limited to any particular mechanism.

miRNAs are expressed in the cell as 100-500 bp precursor RNAs (pre-miRNA), which are processed to form mature ˜70 bp miRNAs. To understand the regulation of genes by miRNAs researchers express either the long pre-miRNA or the mature miRNA. The advantage of expressing either form of miRNA from the promoters of the present invention (e.g., U1 promoter) is that the termination box sequence is statistically less probably to be found in these sequences than the termination sequence of U6 (UUUUU). In fact, a search of the miRNA Registry database of the Wellcome Trust Sanger Institute (Griffiths-Jones, Nucl. Acids Res., 32:D109-D111, 2004), revealed that of 750 miRNAs examined, about half have a UUUU termination sequence and 130 have a UUUUU termination sequence. None were found to have the U1 termination sequence.

In some preferred embodiments, the present invention provides vectors having a pol II snRNA promoter (e.g., U1-5 snRNA promoters). This is in contrast to the U6 promoter, which serves as a pol III promoter. The present invention is not limited by the source or identity of the pol II snRNA promoter. In some embodiments, the vector comprises a human pol II snRNA promoter. The human pol II snRNA core promoters contain only one important element, the proximal sequence promoter (PSE). The distal sequence promoter (DSE) serves to enhance transcription from the core promoter, but is not necessary for function. Numerous references describe and characterize the pol II promoters from various organisms (see e.g., Li et al., J. Virol., 72:4205, 1998; Kwek et al., Nat. Struct. Biol, 9:800, 2002; Abounader et al., Methods Mol. Biol., 252:209, 2004). For illustration purposes, the present invention is described in the context of the U1 promoter, below. The present invention is not limited to such embodiments.

The U1 promoter is a pol II promoter and is recognized by many of the same RNA polymerase II enzyme subunits as are mRNA pol II promoters. This similarity includes recognition by factor TFIIA. RNA polymerase III, in contrast, does not appear to require TFIIA. Inducible systems involved transcription factors that activate RNA polymerases. For example, VP16 is the transcription factor that is part of the tet-on and tet-off system. VP16 functions by interacting with TFIIA to promote transcription. Because the U1 promoter is recognized by TFIIA, inducible systems that utilize transcription factors that interact with TFIIA find use in regulating the U1 promoter. In contrast, the RNA polymerase III promoters are not amenable to this type of regulation.

In some preferred embodiments, the vectors of the invention comprise a pol II snRNA promoter operably associated with an RNAi molecule (e.g., miRNA or siRNA) or RNAi molecule precursor, alone or with other sequences of interest (reporter molecules, etc.). Experiments conducted during the development of the present invention demonstrated the surprising result that expression (at or above 50% of maximum) of the RNAi molecule was prolonged for seven or more days. This is in contrast to prior systems that typically observed expression of about three days. Thus, the present invention provides compositions and methods for prolonged RNAi molecule expression (e.g., 4, 5, 6, 7, or more days).

Experiments conducted during the development of the present invention demonstrated that restriction enzyme sequences may be used between the pol II snRNA promoter and the expressed sequence without interfering with the promoter. Thus, in additional to the variety of other commonly used vector components, cloning or multiplecloning sites may be used freely in the vectors of the present invention.

Contrary to prior art methods, the vectors of the present invention may incorporate the RNAi molecule directly without the need to encode a longer sequence harboring the RNAi molecule. Prior art methods typically embed the RNAi molecule in another gene to allow proper expression. The vectors of the present invention were found to work (transcribe and terminate) well with very short transcribed sequences, obviating the need for the inclusion of other coding sequences. Experiments conducted during the development of the present invention also demonstrated that the vectors having pol II snRNA promoters (e.g., U1 promoter) may be used to express RNAi components that start with a guanosine base. The U1 promoter typically transcribes sequences starting with an adenosine.

In some preferred embodiments, the vectors of the present invention comprise a pol II snRNA termination box (e.g., a U1 termination box when a U1 promoter is used); In particularly preferred embodiments, a spacer separates the termination box from the insert. Experiments conducted during the development of the present invention found that short spacers (e.g., 3 bases) did not work well. A ten base spacer was preferred.

The present invention also provides methods for making and using the vectors and kits of the present invention. For example, the vectors of the present invention, harboring RNAi molecules or precursors may be used in RNA interference experiments in vitro or in vivo, transiently or stably, for research, diagnostic, drug screening, and therapeutic applications. For example, in some embodiments, the vectors comprise viral vectors (e.g., adenovirus, adeno-associated virus, lentivirus, etc.). In addition to increasing transfection efficiency, viral infection may also offer the benefit of transfection of cells not amenable to other transfeciton methods, including primary and neuronal cell lines. In some embodiments, the vectors are used as gene therapy vectors to express an RNAi molecule in an organism for research or therapeutic uses.

In some embodiments, the vectors comprise a sequence that permits inducible expression of the RNAi molecule (see Example 5). In some embodiments, the inducible expression is tissue specific. For example, a tissue specific promoter may be used to initiate expression of a repressor or inducer that interferes with the expression of the RNAi molecule. This system may be included on a separate vector or on the same vector as the RNAi molecule. The inducible system can be incorporated into the promoter with multiple strategies, including, but not limited to, insertion of control elements, replacement of U1 promoter sequences with control elements, or adding control elements to a minimal U1 promoter. In some embodiments, species-specific promoters are used (e.g., human, mouse, rat, etc.). In some embodiments tissue-specific isoforms of a promoter sequence are used (e.g., Caceres et al., Nucleic Acids Research, 20:4247 (1992)).

The present invention also provides kits comprising the above vectors or kits comprising components that permit the assembly of such vectors. For example, in some embodiments, the present invention provides linearized or linearizable vectors comprising a pol II snRNA promoter configured to permit a user to insert an RNAi molecule of interest. For example, in some such embodiments, the user linearizes the vector, mixes with a double-stranded RNAi molecule of interest, and ligates. Thus, the kits, in such embodiments, preferably provide the vector, ligase, ligase buffer, annealing buffer, and control samples (positive and negative controls). The kit may also include instructions. This is particularly the case where the kit is used either as part of an in vitro diagnostic produce or a therapeutic product.

In other kit embodiments, the kits are provided with components that permit a double stranded RNAi expression cassette to be produced by a user using, for example, PCR or other primer extension methods. For example, the vector is provided as a linearized or linearizable vector. In preferred embodiments, the vector is provided as linearized in the kit, having blunt ends and dephosphorylated with calf intestinal alkaline phosphatase to prevent or reduce self-ligation. The kit further contains an amplification template containing pol II snRNA sequence. The kit further contains a primer (e.g., a 5′ primer) that is complementary to the promoter sequence or to one end of a template containing the promoter sequence. The user provides one strand of an RNAi molecule configured to act as an extension primer in the opposite direction of the primer provided in the kit, such that the 3′ end of the RNAi molecule is complementary to a portion of the other end of the pol II snRNA template sequence. PCR amplification of the template in the presence of the primers generates a double stranded product having the pol II snRNA promoter operably associated with the RNAi molecule. This construct is then readily incorporate into the vector. Thus, in some such embodiments, the kit comprises a PCR master mix (i.e., having all of the components needed to carry out a PCR reaction), the vector, the promoter template, and a primer. One skilled in the art will appreciate that the user may provide any one or more of these components without their provision in the kit. In preferred embodiments, a polymerase is used that permits efficient amplification or extension in the presence of a hairpin structure (e.g., a hairpin found in the RNAi molecule). Experiments conducted during the development of the present invention revealed that the Taq polymerase enzyme does not effectively work in these amplification reactions. Further experiments identified Tli polymerase (Promega Corporation, Wisconsin) as an effective enzyme of choice. One skilled in the art will also appreciate various alternative ways of producing such a vector.

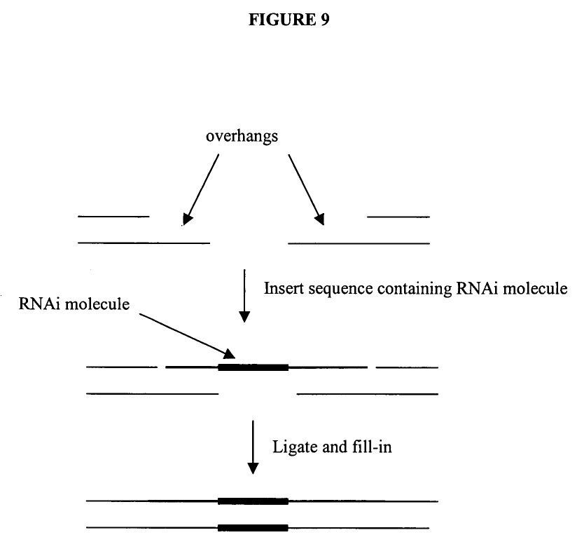

In some embodiments, the present invention provides a kit for use with high-volume cloning of many RNAi molecules. For example, in some embodiments, the kit contains nucleic acid components configured to provide two separate double stranded structures having overhands (e.g., 4-12 base overhands generated by restriction enzyme digestion). FIG. 9 shows one such embodiments. The two components are configured such that a sequence comprising an RNAi molecule (e.g., a hairpin oligonucleotide) hybridizes to each of the overhangs (i.e., the 5′ end of the sequencing having the RNAi molecule hybridizes to one of the overhands, while the 3′ end hybridizes to the other), such that a gap on the complementary strand is generated across from the RNAi molecule sequence. The structure is then ligated and a polymerase or other suitable enzyme fills in the gap to create a double-stranded sequence of a vector containing the sequence comprising the RNAi molecule. Such kits find particular use for the insertion of large numbers of random RNAi sequences. In such embodiments, the ends of the sequence comprising an RNAi molecule are known, so as to allow hybridization and ligation to the overhangs, but the RNAi sequence may be unknown. Large libraries of vectors containing RNAi sequences may thus be generated.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); PCR (polymerase chain reaction); WT (wild type); SN (supernatant); and RS (restriction site).

### Example 1

### Creation of U1 Promoter RNAi Vectors

Control Vector Construction

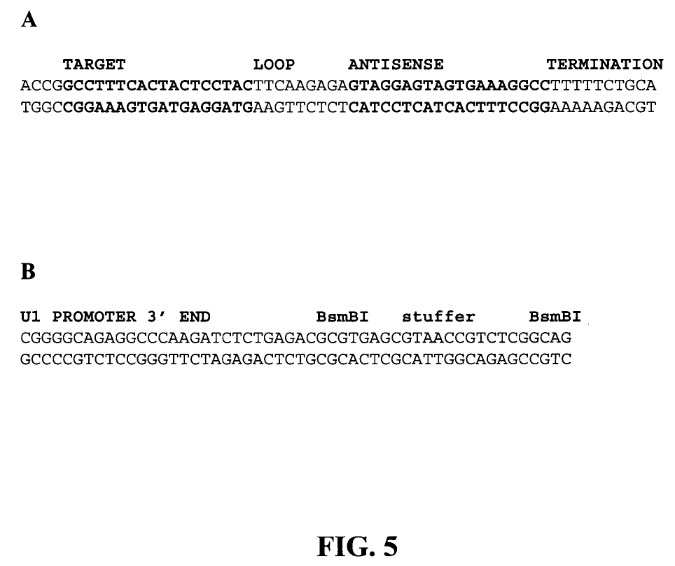

A positive control vector for generation of siRNA hairpin constructs containing a U6 promoter was prepared for inhibition of Renilla luciferase expression using the linearized vectors psiSTRIKE™ Basic Vector and psiSTRIKE™ Puromycin Vector as described in Technical Manual 246 for the siSTRIKE™ U6 Hairpin Cloning System (Promega Corporation). Oligonucleotides A) 5′-ACCGGCCTTT CACTACTCCT ACTTCAAGAG AGTAGGAGTA GTGAAAGGCC TTTTTC-3′ (SEQ ID NO:1) and B) 5′-TGCAGAAAAA GGCCTTTCAC TACTCCTACT CTCTTGAAGT AGGAGTAGTG AAAGGC-3′ (SEQ ID NO:2) were annealed and ligated into the psiSTRIKE™ Basic Vector per instructions yielding the Renilla luciferase hairpin sequence insert of FIG. 5A (SEQ ID NO:17). The highlighted sequences are the nucleotides that will form the hairpin loop structure. The loop is included in the hairpin, but it is not highlighted. A non-specific hairpin construct was also created to serve as a negative control.

A U1 promoter control sequence was prepared to determine if the U1 promoter sequence alone caused inhibition of Renilla luciferase expression. The U1 promoter was amplified out of the pH1U1 vector (kind gift from Dr. James Dahlberg, University of Wisconsin Madison), which contains U1 promoter sequence 40-431 nt from GENBANK Accession No. J00318), using the 5-primer C) 5′-ATCCTAAGGA CCAGCTTCTT TGGG-3′ (SEQ ID NO:3) and the 3′ primer D) 5′-ATCCTGCCGA GACGGTTACG CTCACGCGTC TCAGAGATCT TGGGCCTCTG C-3′ (SEQ ID NO:4). The amplification product was used as a ‘promoter control’ in some experiments as detailed below. The 3′ end of the amplification product is shown in FIG. 5B (SEQ ID NO:18), while the full length amplification product is provided as SEQ ID NO:25 (ATCCTAAGGACCAGCTTCTTTGGGAGAGAACAGACGCAGGGGCGGGAGGGAAAAA GGGAGAGGCAGACGTCACTTCCCCTTGGCGGCTCTGGCAGCAGATTGGTCGGTTGA GTGGCAGAAAGGCAGACGGGGACTGGGCAAGGCACTGTCGGTGACATCACGGACA GGGCGACTTCTATGTAGATGAGGCAGCGCAGAGGCTGCTGCTTCGCCACTTGCTGCT TCACCACGAAGGAGTTCCCGTGCCCTGGGAGCGGGTTCAGGACCGCTGATCGGAAG TGAGAATCCCAGCTGTGTGTCAGGGCTGGAAAGGGCTCGGGAGTGCGCGGGGCAAG TGACCGTGTGTGTAAAGAGTGAGGCGTATGAGGCTGTGTCGGGGCAGAGGCCCAAG ATCTCTGAGACGCGTGAGCGTAACCGTCTCGGCAGATG).

Test Vector Construction

To define the U1 promoter sequence and any nucleotide spacer features useful for optimal hairpin siRNA expression and inhibition of a target gene, several vectors were constructed. Briefly, U1 promoter sequences were incorporated 5′ of the Renilla luciferase hairpin sequence, and spacer nucleotides were inserted between the hairpin loop structure and the termination box region. Vector constructs were generated as instructed in Technical Manual 247 for the siLentGene™-2 U6 Hairpin Cloning System (Promega Corporation), herein incorporated by reference in its entirety, and amplifications were performed using the pH1U1 vector as a template for the U1 promoter. Amplification products were ligated into the psiLentGene™ Basic Vector and the psiLentGene™ Puromycin Vector as described in the technical literature.

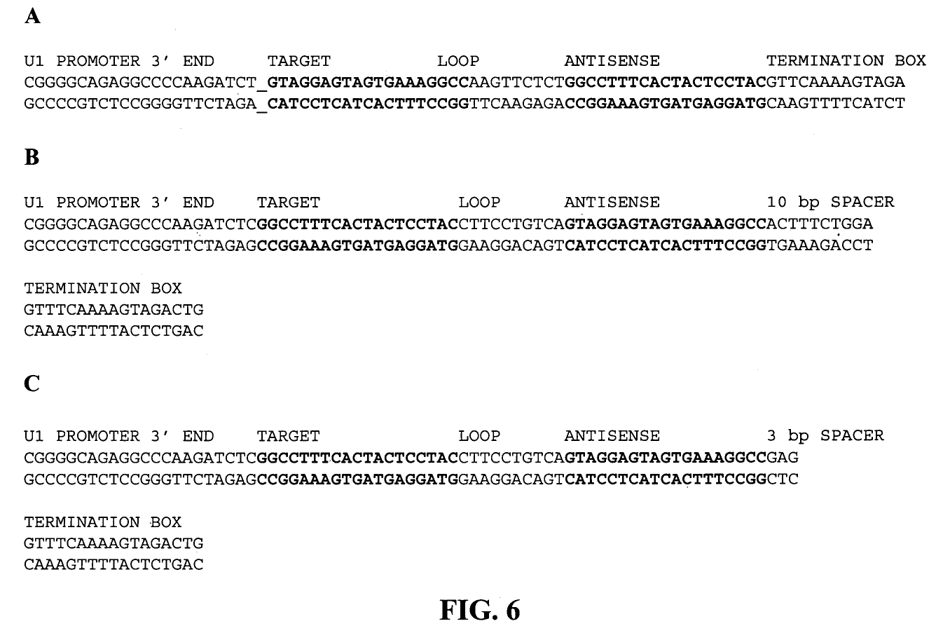

The oligonucleotide pairs used for amplification include pair 1 E) 5′-ATCCTAAGGA CCAGCTTCTT TGGG-3′ (SEQ ID NO:5) and F) 5′-ATCTCTACTT TTGAACGTAG GAGTAGTGAA AGGCCAGAGA ACTTGGCCTT TCACTACTCC TACAGATCTT GGGGCCTCTG CCCCGACAC-3′ (SEQ ID NO:6), which yielded the Renilla luciferase hairpin sequence of FIG. 6A with no incorporated nucleotide spacers between the final hairpin structure and the termination sequence (SEQ ID NO:19). Highlighted sequences are the nucleotides that form the hairpin structure, boxed nucleotides represent an addition to the normal U1 promoter sequence, and the underline represents a deletion of the nucleotides cytosine and its complement guanine. Other nucleotide pairs for amplification include pair 2 G) 5′-ATCCTAAGGA CCAGCTTCTT TGGG-3′ (SEQ ID NO:7) and H) 5′-ATCCAGTCTC ATTTTGAAAC TCCAGAAAGT GGCCTTTCAC TACTCCTACT GACAGGAAGG TAGGAGTAGT GAAAGGCCGA GATCTTGGGC CTCTGC-3′ (SEQ ID NO:8), which yielded the Renilla luciferase hairpin sequence of FIG. 6B with a 10 bp spacer between the final hairpin loop structure and the termination sequence (SEQ ID NO:20). The final nucleotide pairs used for amplification include pair 3 I) 5′-ATCCTAAGGA CCAGCTTCTT TGGG-3′ (SEQ ID NO:9) and J) 5′-ATCCAGTCTC ATTTTGAAAC CTCGGCCTTT CACTACTCCT ACTGACAGGA AGGTAGGAGT AGTGAAAGGC CGAGATCTTG GGCCTCTGC-3′ (SEQ ID NO:10), which yielded the Renilla luciferase hairpin sequence of FIG. 6C with a 3 bp spacer between the final hairpin loop structure and the termination sequence (SEQ ID NO:21).

Cell Transfection

HeLa cells, stably transfected with a Renilla luciferase construct optimized for expression in mammalian cells, were used for testing the U1 promoter constructs. HeLa cells were transfected using the TransIT®-LT1 Transfection Reagent (Mirus Corporation) as described in the product technical literature (ML001, Rev. 1/04). Briefly, cells were plated at 3000 cells/well in 100 μl total volume complete HeLa medium containing DMEM supplemented with 10% FBS and 1% each penicillin and streptomycin in white well, clear bottom 96-well plates (Costar), and incubated overnight at 37° C. in 5% CO₂. The following day, the transfection complex was prepared by adding 36 μl of the TransIT®-LT1 reagent to 3 ml of DMEM and incubating the mixture for 15 minutes at room temperature. Vector DNA was added to the LT1/DMEM mixture at 0.1 μg/well, and the LT1/DMEM/DNA mixture was allowed to incubate an additional 20 minutes at room temperature. Immediately prior to addition of the transfection mixture, media was changed on the cells and 25 μl of the LT1/DMEM/DNA mixture was added to each well containing cells. The same procedure was followed for each different DNA construct. The transfected cells were incubated overnight at 37° C. in 5% CO₂. The day following transfection, the transfection medium was replaced with complete growth medium as previously described.

Two days following transfection, 60 μM of a cell permeable coelentrazine substrate for Renilla luciferase Viviren (Promega Corporation), was added to each well and the cells were incubated at room temperature for 2 minutes. After the 2 minute incubation, luminescence reflecting Renilla luciferase expression was measured on a FLUOstar Optima multi-detection plate reader (BMG Labtech) n=6 and reported as Renilla relative light units (RLU). Cell viability was assayed and reported as firefly luciferase RLU immediately following Renilla luminescence detection by using the CellTiter-Glo® Luminescent Cell Viability Assay as defined in technical literature (TM288, Promega Corporation). To normalize Renilla luciferase mediated luminescence to live cells, Renilla RLUs were divided by the light units generated in the CellTiter-Glo® Luminescent Cell Viability Assay. Further, to determine the amount of Renilla RLUs associated with specific Renilla luciferase inhibition, Renilla specific RLUs were divided by the RLUs generated using the non-specific hairpin control DNA. Reduction of Renilla luminescence is presented as ‘Percent Inhibition’ based on the normalizations.

Results

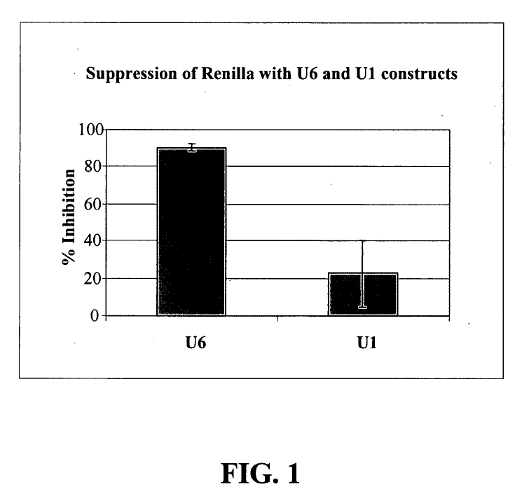

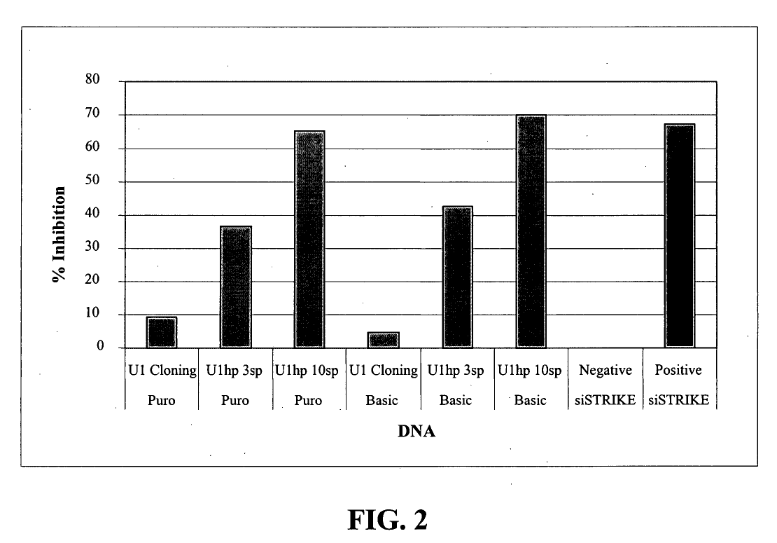

As demonstrated in FIG. 1, when comparing the U6 promoter (primer set A and B and associated construction) to a U1 promoter (primer pair 1 including primers E and F and associated construction) in transcription and subsequent inhibition of Renilla luciferase gene expression, the U1 promoter region with a deleted cytosine at the 3′ end and an added cytosine 8 nt in from the 3′ end, did not inhibit Renilla luciferase expression as well as the U6 promoter of the psiSTRIKE vector system. However, using a U1 promoter region without the addition or deletion of a cytosine, and in association with a ten base pair spacer between the hairpin structure and the termination box, yielded inhibition comparable to that from the U6 promoter as shown in FIG. 2. This inhibition was seen regardless of whether or not the puromycin selectable gene was present in the vector.

### Example 2

### Long Term Transient Expression of Hairpin siRNA in Mammalian Cells

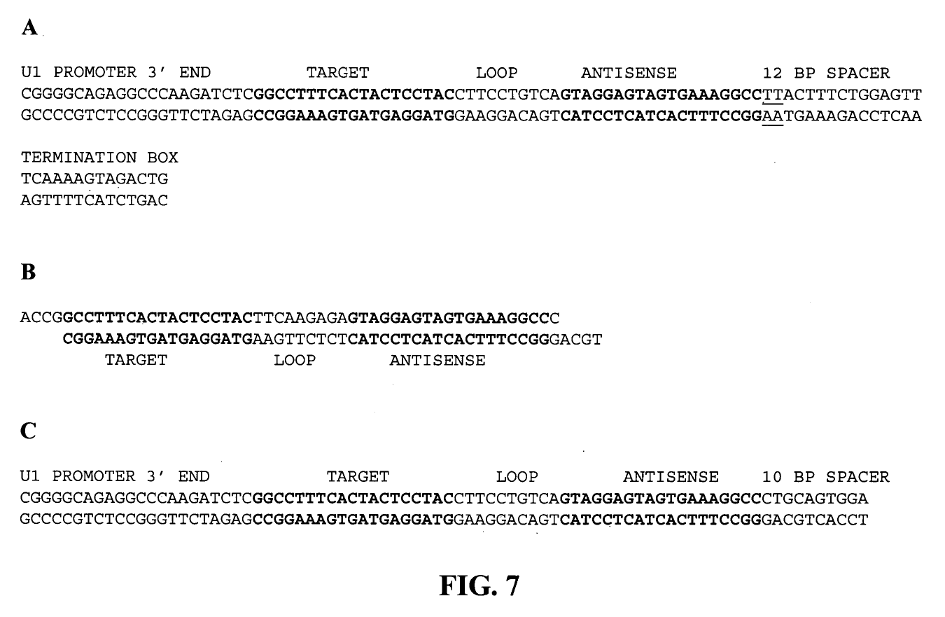

DNA constructs containing hairpin siRNA for inhibition of Renilla luciferase were prepared as previously described. The U1 promoter region used was the same as outlined for the DNA constructs with the 10 bp spacer insert as described in Example 1, and the spacer sequence between the hairpin structure and the termination box was the same as the 10 bp spacer region of Example 1, with the addition of two thymidines added to the 5′ end of the spacer region making a 12 bp spacer. These additional thymidines were added in the spacer region in an attempt to mimic the nucleotide overhangs that DICER produces when cleaving dsRNA. The oligonucleotides used to generate the U1 promoter and siRNA insert included sense primer K) 5′-ATCCTAAGGA CCAGCTTCTT TGGG-3′ (SEQ ID NO:11) and antisense primer L) 5′-ATCCAGTCTC ATTTTGAAAC TCCAGAAAGT AAGGCCTTTC ACTACTCCTA CTGACAGGAA GGTAGGAGTA GTGAAAGGCC GAGATCTTGG GCCTCTGC-3′ (SEQ ID NO:12) yielding the insert of FIG. 7A (SEQ ID NO:22). Highlighted sequences are the nucleotides that will form the target hairpin structure. The loop is part of hairpin, but is not highlighted. The underlined sequences are the additional nucleotides in the spacer region. The U6 promoter construct as described in Example 1 was used for comparison.

Transfections were done as described in Example 1, however cells were transfected at half the cell density (1.75×10⁵ cells/well) to allow growth for 7 days. Samples were transfected in multiple sets to allow harvest and assay of cells at days 3, 5 and 7 post-transfection. Media was changed to complete HeLa medium on days 2, 4, and 6 post-transfection. Assays for Renilla luciferase inhibition and cell viability and normalization calculations were performed on days 3, 5, and 7 post-transfection, also as described in Example 1.

Results

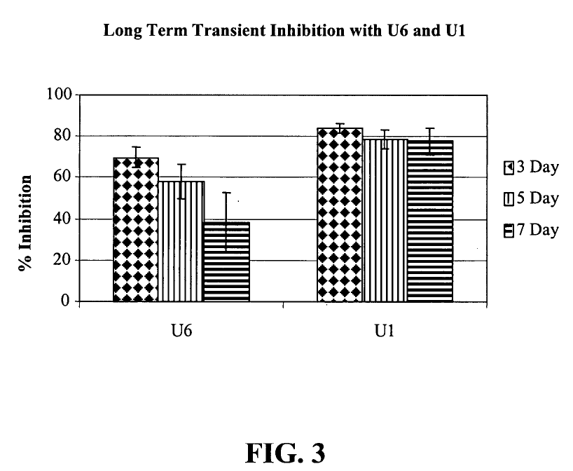

As seen in FIG. 3, long term inhibition of Renilla luciferase expression was seen over a period of 7 days when the U 1 promoter was used for expression of the Renilla hairpin RNA, whereas the U6 promoter construct did not exhibit long term expression over the same time period. Inhibition of expression from the U1 construct remained at about 60% or above for the whole 7 day period, while at day 7 inhibition attributed to the U6 construct was down to about 30%, or half that observed for the U1 construct. Similarly, initial inhibition of Renilla luciferase expression at day 3 post-transfection for the U6 promoter hairpin siRNA was less (mean around 69% inhibition) than that seen for the U1 promoter system (mean around 83% inhibition).

### Example 3

### Hairpin Expression and Requirement for Termination Sequence

DNA constructs containing the U6 promoter and termination sequence and DNA constructs containing the U1 promoter and termination box were prepared as described in Example 1. Additional DNA constructs containing the U6 and the U1 promoters in the absence of termination sequences, were created for comparison purposes. For the U6 promoter construct without a termination sequence, the following primers were used: sense primer M) 5′-ACCGGCCTTT CACTACTCCT ACTTCAAGAG AGTAGGAGTA GTGAAAGGCC C-3′ (SEQ ID NO:13); and antisense primer N) 5′-TGCAGGGCCT TTCACTACTC CTACTCTCTT GAAGTAGGAG TAGTGAAAGG C-3′ (SEQ ID NO:14), yielding the hairpin insert of FIG. 7B (SEQ ID NO:23). The U1 promoter construct without a termination box sequence was created as described in Example 1 using the following primers: sense primer O) 5′-ATCCTAAGGA CCAGCTTCTT TGGG-3′ (SEQ ID NO:15) and antisense primer P) 5′-TACTCCACTG CAGGGCCTTT CACTACTCCT ACTGACAGGA AGGTAGGAGT AGTGAAAGGC CGAGATCTTG GGCCTCTGC-3′ (SEQ ID NO:16), yielding the hairpin insert of FIG. 7C (SEQ ID NO:24).

HeLa cell transfections were performed as described in Example 1. Cells were assayed for Renilla luciferase expression and cell viability two days post-transfection, and normalizations of inhibition to cell viability and non-specific inhibition were calculated as in Example 1.

Results

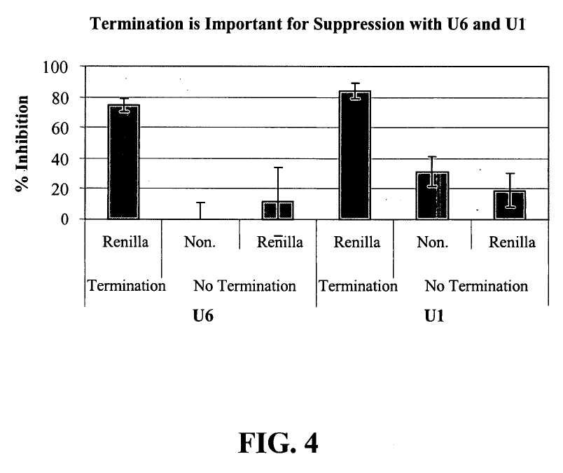

The incorporation of a termination sequence was important for efficient expression of the hairpin siRNA, and subsequent inhibition of Renilla luciferase in HeLa cells, as seen in FIG. 4 regardless of which promoter is used. Inhibition of luciferase expression is 3 times greater when a termination sequence is present as compared to inhibition from a construct lacking a termination sequence.

### Example 4

### Viral Vectors

The efficiency of viral delivery of DNA-directed RNAi circumvents the problems associated with common transfection procedures. Viral delivery includes the benefits of more effective delivery to a broader range of cell types, including non-dividing cells. In some embodiments of the present invention, DNA-directed RNAi including the U1 promoter is packaged in the viral coat to form viral particles for infection of cells. To generate viral particles including the U1 promoter and short hairpin RNA sequences, the U1 promoter is placed in the context of DNA elements that facilitate viral production and antibiotic selection. The U1 promoter is compatible with many virus systems, including, but not limited to, lentiviral, retroviral, adenoviral and plant viruses.

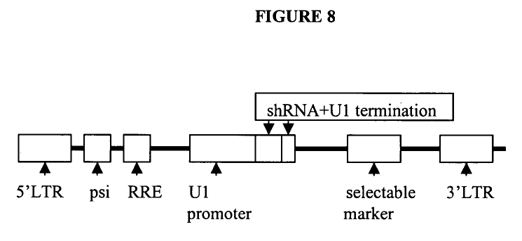

For example, to deliver U1 RNAi with a lentiviral system, the U1 promoter and short hairpin RNA (shRNA) or microRNA (mRNA) sequence followed by the appropriate U1 termination sequence is positioned in a vector after a 5′LTR (long terminal repeat), psi site (cis-acting RNA packaging signal), and RRE (Rev response element), and before a eukaryotic selectable marker, followed by the 3′ LTR (see FIG. 8). The vector also contains appropriate antibiotic markers for bacterial cells.

Co-transfection into appropriate cells (293FT cell line, for example) of packaging vectors and the viral vector containing the U1 promoter and shRNA generates viral particles containing the U1 promoter vector. The virus particles are then added to mammalian cells to transduce. The euckaryotic selectable maker allows selection of transduced mammalian cells.

### Example 5

### Inducible Vectors

The polymerase II U1 promoter contains two predominant upstream elements, the PSE (proximal sequence element) and DSE (distal sequence element), but it does not contain a TATA box. An inducible U1 promoter is generated by placement of regulatable operator(s) close to or inside of the U1 promoter. The presence of the operator(s) decreases the background transcription from the U1 promoter, with transcription increasing with addition of an appropriate inducer.

An example of placement of the tetracycline operators near the PSE is shown below. In the absence of tetracycline, the tetracycline repressor binds the operator and significantly decreases the background levels of transcription from the U1 promoter. In the presence of tetracycline, the repressor is bound by tetracycline and removed from the U1 promoter, thereby relieving repression of transcription. The tetracycline repressor is expressed in cells from either a transient transfection of a plasmid containing the tetracycline repressor gene or in a stable cell line expressing the tetracycline repressor.

As shown in the sequence below, the U1 promoter with a tet operator may be positioned before the PSE. The sequence begins with the U1 promoter at nucleotide −87. The promoter would include nucleotides (−392 to +1). Uppercase letters indicate the tet operator (tet O1) and the bold represents the U1 PSE. A gene follows the promoter (luciferase, for example) that allows monitoring of the operator and inducer effects.

(SEQ ID NO:26)

CtcgggagtgcgcggggcACTCTATCATTGATAGAGTaagtgaccgtgtg
tgtaaagagtgaggcgtatgaggctgtgtcggggcagaggcccaaga
tctc

In some embodiments, the U1 promoter with a tet operator is positioned before the PSE. The sequence below begins with U1 promoter at nucleotide −87. The promoter would include nucleotides (−392 to +1). Uppercase letters indicate the tet operator (tet O2) and the bold represents the U1 PSE.

(SEQ ID NO:27)

CtcgggagtgcgcggggcCTCCCTATCAGTGATAGAGAAAGTGaagtgac
cgtgtgtgtaaagagtgaggcgtatgaggctgtgtcggggcagaggccca
agatctc

In some embodiments, the U1 promoter with a tet operator is positioned after the PSE. The sequence below begins with the U1 promoter at nucleotide −87. The promoter would include nucleotides (−392 to +1). Uppercase letters indicate the tet operator (tet O1) and the bold represents the U1 PSE.

(SEQ ID NO:28)

Ctcgggagtgcgcggggcaagtgaccgtgtgtgtaaagagtgaggcgtat
gaACTCTATCATTGATAGAGTatctc

In some embodiments, the U1 promoter with a tet operator is positioned after the PSE. The sequence below begins with the U1 promoter at nucleotide −87. The promoter would include nucleotides (−392 to +1). Uppercase letters indicate the tet operator (tet O2) and the bold represents the U1 PSE:

(SEQ ID NO:29)

Ctcgggagtgcgcggggcaagtgaccgtgtgtgtaaagagtgaggcgtat
gaCTCCCTATCAGTGATAGAGAAAGTGatctc

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant fields are intended to be within the scope of the following claims.

We claim:

1. A composition comprising a vector, said vector comprising an snRNA pol II promotor operably associated with an RNAi molecule.

2. The composition of claim 1, wherein said snRNA pol II promoter comprises a U1 promoter.

3. The composition of claim 1, wherein said snRNA pol II promoter comprises a U2 promoter.

4. The composition of claim 1, wherein said RNAi molecule comprises a sequence encoding an siRNA.

5. The composition of claim 1, wherein said RNAi molecule comprises a sequence encoding an miRNA.

6. The composition of claim 1, wherein said vector further comprises an snRNA pol II termination sequence.

7. The composition of claim 6, wherein said termination sequence is separated from said promoter by a nucleic acid linker.

8. The composition of claim 6, wherein said vector further comprises a restriction enzyme cloning site between said promoter and said termination sequence.

9. The composition of claim 1, wherein said vector comprises a viral vector sequence.

10. The composition of claim 1, wherein said vector further comprises a sequence that permits inducible expression of said RNAi molecule.

11. A composition comprising a host cell comprising the vector of claim 1.

12. The composition of claim 11, wherein said cell is in culture.

13. The composition of claim 11, wherein said cell resides in vivo.

14. The composition of claim 11, wherein said vector is stably integrated into the genome of said cell.

15. A method for gene expression silencing, comprising the step of transfecting a cell with the vector of claim 1.

16. A kit for cloning an RNAi molecule, comprising: i) an snRNA RNA polymerase II promoter template oligonucleotide, ii) a primer complementary to said template, iii) a vector, iv) amplification reagents, and v) ligation reagents.

17. The kit of claim 16, wherein said promoter comprises a U1 promoter.

18. The kit of claim 16, wherein said vector comprises a blunt ended vector.

19. The kit of claim 16, further comprising a RNAi molecule.

20. The kit of claim 16, wherein said amplification reagents comprise a high fidelity proof reading DNA polymerase.

21. The kit of claim 20, wherein said polymerase comprises Tli polymerase.

22. The kit of claim 21, wherein said kit further comprises an amplification buffer comprising magnesium sulphite.

23. The kit of claim 16, wherein said vector is linearized.

24. The kit of claim 16, wherein said vector has been treated with a phosphatase.

25. A kit comprising a vector, said vector comprising an snRNA pol II promotor operably associated with an RNAi molecule.

26. The kit of claim 25, further comprising ligation reagents.

27. The kit of claim 25, wherein said promoter is a U1 promoter.
